(19) United States
(12) Patent Application Publication
(10) Pub. No.: US 2016/0354395 A1
(43) Pub. Date: Dec. 8, 2016

(54) INFANT FORMULAS CONTAINING A 2-FUCOSYLATED OLIGOSACCHARIDE FOR TREATMENT OR PREVENTION OF INFLUENZA INFECTION

(71) Applicant: NESTEC S.A., Vevey (CH)

(72) Inventors: Nikhat Contractor, Bryn Mawr, PA (US); Martin Jon Kullen, Pottstown, PA (US)

(73) Assignee: NESTEC S.A., Vevey (CH)

(21) Appl. No.: 15/036,841

(22) PCT Filed: Nov. 14, 2014
(86) PCT No.: PCT/EP2014/074558

§ 371 Date: May 16, 2016

Related U.S. Application Data
(60) Provisional application No. 61/904,571, filed on Nov. 15, 2013.

(51) Int. Cl. A61K 31/702; A23L 33/21; A23L 33/00; A61K 9/00; A61K 31/733

(52) U.S. Cl. CPC A61K 31/702; A61K 9/0095; A61K 31/733; A23L 33/40; A23L 33/21; A23V 2002/00

(57) ABSTRACT

Compositions for use in the treatment or prevention of influenza infection are provided, and the compositions contain a 2-fucosylated oligosaccharide, such as 2′-fucosyllactose (2′FL). The compositions can also include fructooligosaccharides (FOS). Additionally said compositions can be used for improving resistance to an influenza virus, decreasing influenza in the lung, decreasing influenza-induced anorexia, decreasing influenza-induced pulmonary mucin secretion, and decreasing influenza-induced IgG response. They may be used in a infant or a young child.

### BACKGROUND

The present disclosure generally relates to methods and compositions for treatment or prevention of influenza infection. More specifically, the present disclosure relates to compositions comprising a 2-fucosylated oligosaccharide, such as 2′-fucosyllactose (2′FL), and methods comprising administering such compositions.

An influenza infection (also known as flu) is a contagious respiratory illness caused by viruses. Influenza is usually an acute, self-limited respiratory tract infection that begins with the sudden onset of high fever, followed by inflammation of the upper respiratory tree and trachea, with coryza, cough, headache, prostration, malaise and other signs and symptoms that persist for 7-10 days.

The virus replicates in both the upper and lower respiratory tract. In experimental infections in healthy volunteers, influenza A viral replication peaks approximately 48 hours after inoculation into the nasopharynx, declining thereafter, with usually little or no virus shed after six days. As with other respiratory viruses, influenza viruses can cause more severe infections in infants, the elderly and immunodeficient persons. In those individuals whose immune system is compromised or not fully developed, such as infants and young children, influenza-associated disease will often lead to severe viral pneumonitis or be complicated by bacterial superinfection, leading to pneumonia and sepsis.

Seasonal influenza also occasionally results in neurologic complications. Even in non-pandemic years, more than 40,000 deaths annually are attributable to influenza infections in the United States. Strategies to prevent an influenza infection or reduce the symptoms are limited.

Vaccination is the most well-known example. However, protection after vaccination varies from moderate to high. Especially in elderly people with a compromised immune reactivity, protection after vaccination is not optimal. The apparent variation in efficacy is due to a number of factors, including vaccine immunogenicity and the degree of match between the vaccine strain chosen before the influenza season and circulating virus strain(s). Besides vaccination, an alternative strategy to combat influenza infection would be to increase resistance by nutritional components. This should preferably be achieved without excessive inflammatory responses that can harm host tissue.

Furthermore, although mother's milk is recommended for all infants, in some cases breast feeding is inadequate or unsuccessful for medical reasons or the mother chooses not to breast feed. Nutritional compositions such as infant formulas have been developed for these situations. Accordingly, there is a need for a nutritional composition such as an infant formula for the treatment or prevention of influenza infections which may be conveniently and safely administered.

There is a further need to deliver such health benefit in a manner that is particularly suitable for young subjects such as infants or young children, in a manner that does not involve a classical pharmaceutical intervention, as these infants or young children are particularly fragile. There is a need to deliver such health benefits in a manner that does not induce side effects and/or in a manner that is easy to deliver and well accepted by the parents or health care practitioners. There is a need to deliver such benefits in a manner that keeps the cost of such delivery reasonable and affordable by most.

### SUMMARY

The present inventors surprisingly and unexpectedly found that administration of 2′-fucosyllactose improves resistance to influenza. 2′-fucosyllactose decreases influenza in the lung and influenza-induced anorexia, IgG response, and pulmonary mucin secretion. Accordingly, the present disclosure provides compositions comprising a 2-fucosylated oligosaccharide, such as 2′-fucosyllactose, for treatment or prevention of influenza infection and also provides methods comprising administering a 2-fucosylated oligosaccharide, such as 2′-fucosyllactose, for treatment or prevention of influenza infection.

The present disclosure also provides compositions comprising a 2-fucosylated oligosaccharide, such as 2′-fucosyllactose, for use in improving resistance to an influenza virus in an individual, and particularly in infants and young children. Furthermore, the present disclosure relates to the use of a 2-fucosylated oligosaccharide, such as 2′-fucosyllactose, in the preparation of a composition to be administered in an individual, and particularly in infants and young children, for use in improving resistance to an influenza virus.

The present disclosure also provides compositions comprising a 2-fucosylated oligosaccharide, such as 2′-fucosyllactose, for use in decreasing influenza in a lung of an individual, and particularly in infants and young children. Furthermore, the present disclosure relates to the use of a 2-fucosylated oligosaccharide, such as 2′-fucosyllactose, in the preparation of a composition to be administered in an individual, and particularly in infants and young children, for use in decreasing influenza in a lung of an individual, and particularly in infants and young children.

The present disclosure also provides compositions comprising a 2-fucosylated oligosaccharide, such as 2′-fucosyllactose, for use in decreasing influenza-induced anorexia in an individual, and particularly in infants and young children. Furthermore, the present disclosure relates to the use of a 2-fucosylated oligosaccharide, such as 2′-fucosyllactose, in the preparation of a composition to be administered in an individual, and particularly in infants and young children, for use in decreasing influenza-induced anorexia.

The present disclosure also provides compositions comprising a 2-fucosylated oligosaccharide, such as 2′-fucosyllactose, for use in decreasing pulmonary mucin secretion in an individual, and particularly in infants and young children. Furthermore, the present disclosure relates to the use of a 2-fucosylated oligosaccharide, such as 2′-fucosyllactose, in the preparation of a composition to be administered in an individual, and particularly in infants and young children, for use in decreasing pulmonary mucin secretion.

The present invention disclosure also provides compositions comprising a 2-fucosylated oligosaccharide, such as 2′-fucosyllactose, for use in decreasing influenza-induced IgG response in an individual, and particularly in infants and young children. Furthermore, the present disclosure relates to the use of a 2-fucosylated oligosaccharide, such as 2′-fucosyllactose, in the preparation of a composition to be administered in an individual, and particularly in infants and young children, for use in decreasing influenza-induced IgG response.

It should be noted that all these above-mentioned different uses (or methods) can be obtained separately or preferably in addition of the treatment or prevention of influenza infections.

The present invention disclosure also provides compositions comprising a 2-fucosylated oligosaccharide, such as 2′-fucosyllactose, for use in treating or preventing influenza infections in an individual, and particularly in infants and young children. Furthermore, the present disclosure provides the use of a 2-fucosylated oligosaccharide, such as 2′-fucosyllactose, in the preparation of a composition to be administered in an individual, and particularly in infants and young children, for use in treating or preventing influenza infections.

In a general embodiment, the present disclosure provides a method for treating an influenza infection. The method comprises administering to an individual having the influenza infection a nutritional composition comprising a 2-fucosylated oligosaccharide. In a related embodiment, the 2-fucosylated oligosaccharide is 2′-fucosyllactose.

In an embodiment, the nutritional composition comprises at least 0.1 g of the 2-fucosylated oligosaccharide/100 g of the composition.

In an embodiment, the nutritional composition comprises a prebiotic in an amount of from 0.3 to 10% by weight of the composition. In a related embodiment, the prebiotic comprises fructooligosaccharides (FOS).

In an embodiment, the individual is an infant or a young child.

In an embodiment, the nutritional composition is a synthetic nutritional composition, which means a mixture obtained by chemical and/or biological means, which can be chemically identical to the mixture naturally occurring in mammalian milks (i.e. the synthetic composition is not breast milk).

In an embodiment, the nutritional composition is selected from the group consisting of an infant formula, a starter infant formula, a follow-on formula, a baby food, an infant cereal composition and a fortifier.

In an embodiment, the influenza infection is by a virus selected from the group consisting of Influenzavirus A, Influenzavirus B, Influenzavirus C, Isavirus, Thogotovirus, and combinations thereof.

In another embodiment, a method for preventing an influenza infection is provided. The method comprises administering to an individual a nutritional composition comprising a 2-fucosylated oligosaccharide. In a related embodiment, the nutritional composition comprises fructooligosaccharides (FOS). In a related embodiment, the 2-fucosylated oligosaccharide is 2′-fucosyllactose.

In another embodiment, a method for improving resistance to an influenza virus in an individual is provided. The method comprises administering to the individual a nutritional composition comprising a 2-fucosylated oligosaccharide. In a related embodiment, the 2-fucosylated oligosaccharide is 2′-fucosyllactose.

In another embodiment, a method for decreasing influenza in a lung of an individual is provided. The method comprises administering to the individual a nutritional composition comprising a 2-fucosylated oligosaccharide. In a related embodiment, the 2-fucosylated oligosaccharide is 2′-fucosyllactose.

In another embodiment, a method for decreasing influenza-induced anorexia in an individual is provided. The method comprises administering to the individual a nutritional composition comprising a 2-fucosylated oligosaccharide. In a related embodiment, the 2-fucosylated oligosaccharide is 2′-fucosyllactose.

In another embodiment, a method for decreasing influenza-induced pulmonary mucin secretion in an individual is provided. The method comprises administering to the individual a nutritional composition comprising a 2-fucosylated oligosaccharide. In a related embodiment, the 2-fucosylated oligosaccharide is 2′-fucosyllactose.

In another embodiment, a method for decreasing influenza-induced IgG response in an individual is provided. The method comprises administering to the individual a nutritional composition comprising a 2-fucosylated oligosaccharide. In a related embodiment, the 2-fucosylated oligosaccharide is 2′-fucosyllactose.

In another embodiment, a nutritional composition for treating or preventing an influenza infection is provided. The nutritional composition comprises at least 0.1 g of a 2-fucosylated oligosaccharide/100 g of the composition. In a related embodiment, the 2-fucosylated oligosaccharide is 2′-fucosyllactose.

An advantage of the present disclosure is to use a 2-fucosylated oligosaccharide, such as 2′-fucosyllactose (2′FL), to treat an influenza infection.

Another advantage of the present disclosure is to use a 2-fucosylated oligosaccharide, such as 2′FL, to prevent an influenza infection.

Still another advantage of the present disclosure is to use a 2-fucosylated oligosaccharide, such as 2′FL, in combination with fructooligosaccharides (FOS) to treat or prevent an influenza infection.

Yet another advantage of the present disclosure is to treat or prevent an influenza infection in an infant or a young child.

An additional advantage of the present disclosure is to use a nutritional composition to treat or prevent an influenza infection.

Another advantage of the present disclosure is to provide a method for treating or preventing an influenza infection which does not rely on antibiotics.

Still another advantage of the present disclosure is to treat or prevent an influenza infection without excessive inflammatory responses.

Additional features and advantages are described herein, and will be apparent from, the following Detailed Description and the Figures.

### BRIEF DESCRIPTION OF THE FIGURES

FIG. 1 shows the chemical structure of 2′-fucosyllactose (2′FL).

FIG. 2 is a table containing a description of the experimental and control groups used in the experimental study described in the non-limiting example.

FIG. 3 is a table indentifying the compositions of the experimental diets used in the experimental study.

FIG. 4 is a table containing the statistical analyses of parameters measured in the experimental study.

FIG. 5 is a graph of the effect of fructooligosaccharides (FOS), lactoferrin and 2′-fucosyllactose on food intake after nasal inoculation with 2*104 plaque-forming units (PFU) of a rat-adapted influenza A (H3N2) virus on day 0. Results are presented as mean.

FIG. 6 is a graph of the effect of fructooligosaccharides (FOS), lactoferrin and 2′-fucosyllactose on food intake after nasal inoculation with 2*104 plaque-forming units (PFU) of a rat-adapted influenza A (H3N2) virus on day 0. Results are presented as mean±SEM. Delta food intake was measured as the decrease in food intake during the entire influenza infection (10 days) corrected for the food intake at baseline (day 0). From left to right, the bars represent Control, FOS, Lactoferrin, and 2′FL.

FIG. 7 is a graph of the effect of fructooligosaccharides (FOS), lactoferrin and 2′-fucosyllactose on bodyweight after nasal inoculation with 2*104 plaque-forming units (PFU) of a rat-adapted influenza A (H3N2) virus on day 0. Results are presented as mean±SEM.

FIG. 8 is a graph of the effect of fructooligosaccharides (FOS), lactoferrin and 2′-fucosyllactose on spleen weight after nasal inoculation with 2*104 plaque-forming units (PFU) of a rat-adapted influenza A (H3N2) on day 0. Results are presented as mean±SEM. From left to right for each day, the bars represent Control, FOS, Lactoferrin, and 2′FL.

FIG. 9 is a graph of the effect of fructooligosaccharides (FOS), lactoferrin and 2′-fucosyllactose on lung weight after nasal inoculation with 2*104 plaque-forming units (PFU) of a rat-adapted influenza A (H3N2) on day 0. Results are presented as mean±SEM. From left to right for each day, the bars represent Control, FOS, Lactoferrin, and 2′FL.

FIGS. 10 (10A and 10B) are graphs of the effect of fructooligosaccharides (FOS), lactoferrin and 2′-fucosyllactose on influenza-induced mucin response on day 3 and day 10. Rats received a nasal inoculation with 2*104 plaque-forming units (PFU) of a rat-adapted influenza A (H3N2) on day 0. Lung mucins are expressed as nmol oligosaccharide equivalents per lung. Results are presented as mean±SEM. From left to right in the bottom panel, the bars represent Control, FOS, Lactoferrin, and 2′FL.

FIG. 11 is a graph of the effect of fructooligosaccharides (FOS), lactoferrin and 2′-fucosyllactose on numbers of influenza particles in the lung (log PFU/g lung) three days after nasal inoculation with 2*104 plaque-forming units (PFU) of a rat-adapted influenza A (H3N2. Results are presented as mean±SEM. From left to right, the bars represent Control, FOS, Lactoferrin, and 2′FL.

FIG. 12 is a graph of the effect of fructooligosaccharides (FOS), lactoferrin and 2′-fucosyllactose on numbers of influenza particles in the lung (log PFU/lung) three days after nasal inoculation with 2*104 plaque-forming units (PFU) of a rat-adapted influenza A (H3N2). Results are presented as mean±SEM. From left to right, the bars represent Control, FOS, Lactoferrin, and 2′FL.

FIGS. 13 (13A and 13B) are graphs of the effect of fructooligosaccharides (FOS), lactoferrin and 2′-fucosyllactose on serum IgG 10 days after nasal inoculation with 2*104 plaque-forming units (PFU) of a rat-adapted influenza A (H3N2). Results are presented as mean±SEM. From left to right in the bottom panel, the bars represent Control, FOS, Lactoferrin, and 2′FL.

FIG. 14 is a schematic drawing of major core types of mucin O-glycans.

### DETAILED DESCRIPTION

The different details and embodiments provided in this application apply to every subject matter, i.e. whatever the wording is sometimes “composition for use in” or some other times “method of”. For example, the different details and embodiments described in the paragraphs referring to “the method of the present invention” also apply to the parts drafted under the form “nutritional composition for use in”, and vice versa.

All percentages expressed herein are by weight of the total weight of the composition unless expressed otherwise. When reference is made to the pH, values correspond to pH measured at 25° C. with standard equipment. As used in this disclosure and the appended claims, the singular forms “a,” “an” and “the” include plural referents unless the context clearly dictates otherwise. As used herein, “about” is understood to refer to numbers in a range of numerals. Moreover, all numerical ranges herein should be understood to include all integers, whole or fractions, within the range. The food composition disclosed herein may lack any element that is not specifically disclosed herein. Thus, “comprising” includes “consisting essentially of” and “consisting of.”

As used herein, “infant” means a child under the age of 12 months. The expression “young child” means a child aged between one and three years, also called a toddler.

“Animal” includes, but is not limited to, mammals, which includes but is not limited to, rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage. As used herein, the term “patient” is understood to include an animal, especially a mammal, and more especially a human that is receiving or intended to receive treatment, as treatment is herein defined. While the terms “individual” and “patient” are often used herein to refer to a human, the present disclosure is not so limited. Accordingly, the terms “individual” and “patient” refer to any animal, mammal or human, having or at risk for a medical condition that can benefit from the treatment.

“Nutritional compositions,” as used herein, are understood to include any number of optional additional ingredients, including conventional food additives, for example one or more acidulants, additional thickeners, buffers or agents for pH adjustment, chelating agents, colorants, emulsifiers, excipients, flavor agents, minerals, osmotic agents, a pharmaceutically acceptable carrier, preservatives, stabilizers, sugars, sweeteners, texturizers, and/or vitamins.

The optional ingredients can be added in any suitable amount. The nutritional composition is usually to be taken enterally, orally, parenterally or intravenously, and it usually includes a lipid or fat source and a protein source. It may be, for example, an infant formula, a starter infant formula, a follow-on formula, a baby food, an infant cereal composition, a fortifier or a supplement. The nutritional composition may be in a solid form (e.g. a powder that may be later reconstituted for its consumption) or a liquid.

The expression “infant formula” means a food composition intended for particular nutritional use by infants during the first four to six months of life and satisfying by itself the nutritional requirements of this category of person (Article 1.2 of the European Commission Directive 91/321/EEC of May 14, 1991 on infant formulae and follow-on formulae).

The expression “starter infant formula” means a foodstuff intended for particular nutritional use by infants during the first four months of life.

The expression “follow-on formula” means a foodstuff intended for particular nutritional use by infants aged over four months or by young children, and constituting the principal liquid element in the progressively diversified diet of this category of person.

The expression “baby food” means a foodstuff intended for particular nutritional use by infants or by young children during the first years of life.

The expression “infant cereal composition” means a foodstuff intended for particular nutritional use by infants or by young children during the first years of life. The expression “fortifier” refers to liquid or solid nutritional compositions suitable for mixing with breast milk or infant formula.

“Probiotic” means microbial cell preparations or components of microbial cells with a beneficial effect on the health or well-being of the host (Salminen S, Ouwehand A. Benno Y. et al. “Probiotics: how should they be defined.” Trends Food Sci. Technol. 1999: 10 107-10).

The term “oligosaccharide” means a carbohydrate having a degree of polymerization (DP) ranging from 2 to 20 inclusive but not including lactose.

As used herein, “therapeutically effective amount” is an amount that prevents a deficiency, treats a disease or medical condition in an individual or, more generally, reduces symptoms, manages progression of the diseases or provides a nutritional, physiological, or medical benefit to the individual.

“Prevention” includes reduction of risk and/or severity of influenza infections. As used herein, the terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, such as nitrogen imbalance or muscle loss. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

“2-fucosylated oligosaccharides” and “fucosylated oligosaccharide comprising a 2′ fucosyl-epitope” can be used interchangeably. These terms encompass fucosylated oligosaccharides with a certain homology of form since they contain a 2′-fucosyl-epitope, therefore a certain homology of function can be expected. The 2-fucosylated oligosaccharide can be, for example, selected from the list comprising 2′-fucosyllactose, difucosyllactose, lacto-N-fucopentaose, lacto-N-fucohexaose, lacto-N-difucohexaose, fucosyllacto-N-hexaose, fucosyllacto -N-neohexaose, difucosyllacto-N-hexaose difuco-lacto-N-neohexaose, difucosyllacto-N-neohexaose, fucosyl-para-Lacto-N-hexaose and any combination thereof. In some particular embodiments, the 2-fucosylated oligosaccharide is 2′-fucosyllactose, also abbreviated 2-FL, 2FL, 2′FL, or 2′-FL.

Without wishing to be bound by theory, the inventors believe that 2-fucosylated oligosaccharide supplementation can treat or prevent influenza infections by at least one of the following mechanisms: altering mucin composition and thereby enhancing binding of influenza to mucins, altering microbiota composition and thereby modulating the immune defenses, decreasing bacterial overgrowth in lungs after influenza infection, and/or modulating cytokine profiles and thereby improving the immune response to influenza. Furthermore, 2-fucosylated oligosaccharides may also protect against bacterial pathogens as well.

The nutritional composition provided by the present disclosure comprises a 2-fucosylated oligosaccharide. The 2-fucosylated oligosaccharide may be isolated by chromatographic or filtration technology from a natural source such as animal milks. Alternatively, the 2-fucosylated oligosaccharide may be produced by biotechnology using specific fucosyltransferases and/or fucosidases either by enzyme based fermentation technology (recombinant or natural enzymes) or by microbial fermentation technology. In the latter case, microbes may either express their natural enzymes and substrates or may be engineered to produce respective substrates and enzymes. Single microbial cultures and/or mixed cultures may be used. Fucosylated oligosaccharide formation can be initiated by acceptor substrates starting from any degree of polymerisation (DP) from DP=1 onwards. Alternatively, fucosylated oligosaccharides may be produced by chemical synthesis starting with lactose and free fucose. Fucosylated oligosaccharides are also available for example from Kyowa Hakko Kogyo of Japan.

In an embodiment, an influenza infection is treated or prevented by administering the nutritional composition comprising a 2-fucosylated oligosaccharide to an individual in need of same. For example, the nutritional composition comprising a 2-fucosylated oligosaccharide can be administered to an individual having an influenza infection to treat the influenza infection. The influenza infection can be Influenzavirus A, Influenzavirus B, Influenzavirus C, Isavirus, Thogotovirus, and combinations thereof The nutritional composition can comprise a therapeutically effective amount of the 2-fucosylated oligosaccharide to treat or prevent an influenza infection. In a preferred embodiment, the 2-fucosylated oligosaccharide is 2′-fucosyllactose.

The nutritional composition is preferably an infant formula and preferably is administered to an infant. In an embodiment, the nutritional composition comprises at least 0.1 g of the 2-fucosylated oligosaccharide/100g of the composition on a dry weight basis, preferably from 0.1 to 3 g of the 2-fucosylated oligosaccharide/100g of the composition, and more preferably from 1 to 3 g of the 2-fucosylated oligosaccharide/100g of the composition. The daily dose of the 2-fucosylated oligosaccharides is typically from 0.1 to 4 g.

The nutritional composition can further contain a probiotic. The probiotic microorganisms most commonly used are principally bacteria and yeasts of the following genera: Lactobacillus spp., Streptococcus spp., Enterococcus spp., Bifidobacterium spp. and Saccharomyces spp. In some embodiments, the nutritional composition can further contain a probiotic chosen from probiotic bacterial strains; preferably the probiotic is a Lactobacillus and/or a Bifidobacterium. In an embodiment, the probiotic is at least one of Lactobacillus rhamnosus or Bifidobacterium lactis. Suitable probiotic bacterial strains include Lactobacillus rhamnosus ATCC 53103 available from Valio Oy of Finland under the trademark LGG, Lactobacillus rhamnosus CGMCC 1.3724, Lactobacillus paracasei CNCM 1-2116, Lactobacillus johnsonii CNCM 1-1225, Streptococcus salivarius DSM 13084 sold by BLIS Technologies Limited of New Zealand under the designation KI2, Bifidobacterium lactis CNCM 1-3446 sold inter alia by the Christian Hansen company of Denmark under the trademark Bb 12, Bifidobacterium longum ATCC BAA-999 sold by Morinaga Milk Industry Co. Ltd. of Japan under the trademark BB536, Bifidobacterium breve sold by Danisco under the trademark Bb-03, Bifidobacterium breve sold by Morinaga under the trade mark M-16V, Bifidobacterium infantis sold by Procter & Gamble Co. under the trademark Bifantis and Bifidobacterium breve sold by Institut Rosell (Lallemand) under the trademark R0070.

The nutritional composition can contain a protein source. If the composition is an infant formula, the protein source can be in the composition in an amount of not more than 3.7 or 2.0 g/100 kcal, preferably 1.8 to 2.0 g/100 kcal. The type of protein is not believed to be critical, provided that the minimum requirements for essential amino acid content are met and satisfactory growth is ensured. However, in an embodiment, more than 50% or more than 60% by weight of the protein source is whey (hence ensuring a best balanced amino-acid profile). Thus, protein sources based on whey, casein and mixtures thereof may be used as well as protein sources based on soy. Regarding whey proteins, the protein source may be based on acid whey or sweet whey or mixtures thereof and may include alpha-lactalbumin and beta-lactoglobulin in whatever proportions are desired.

The protein source can be based on modified sweet whey. Sweet whey is a readily available by-product of cheese making and is frequently used in the manufacture of infant formulas based on cows' milk. However, sweet whey includes a component which is undesirably rich in threonine and poor in tryptophan called caseino-glyco-macropeptide (CGMP). Removal of the CGMP from sweet whey results in a protein with a threonine content closer to that of human milk. This modified sweet whey can then be supplemented with those amino acids in respect of which it has a low content (principally histidine and tryptophan). Using modified sweet whey as the principal protein in the protein source enables all essential amino acids to be provided at a protein content between 1.8 and 2.0 g/100 kcal. Such protein sources have been shown in animal and human studies to have a protein efficiency ratio, nitrogen digestibility, biological value and net protein utilization comparable to standard whey-adapted protein sources with a much higher protein content per 100 kcal and to result in satisfactory growth despite their reduced protein content. If modified sweet whey is used as the protein source, it can be supplemented by free histidine in an amount from 0.1 to 1.5% by weight of the protein source.

The proteins may be intact, hydrolyzed or a mixture of intact and hydrolyzed proteins. Partially hydrolyzed proteins (degree of hydrolysis between 2 and 20%) may be used, for example for infants or children believed to be at risk of developing cows' milk allergy. If hydrolyzed proteins are required, the hydrolysis process may be carried out as desired and as known in the art. For example, a whey protein hydrolysate may be prepared by enzymatically hydrolyzing the whey fraction in one or more steps. If the whey fraction used as the starting material is substantially lactose free, the protein suffers much less lysine blockage during the hydrolysis process. This enables the extent of lysine blockage to be reduced from about 15% by weight of total lysine to less than about 10% by weight of lysine, for example about 7% by weight of lysine which greatly improves the nutritional quality of the protein source.

The nutritional composition can contain a carbohydrate source. Any carbohydrate source conventionally found in nutritional compositions for infants and young children like infant formulas, such as lactose, saccharose, maltodextrin, starch and mixtures thereof, may be used, although the preferred source of carbohydrates is lactose. If the composition is an infant formula, preferably the carbohydrate source contributes between 35 and 65% of the total energy of the composition.

The nutritional composition can contain a source of lipids. Preferred fat sources include palm olein, high oleic sunflower oil and high oleic safflower oil. The essential fatty acids linoleic and α-linolenic acid may also be added as may small amounts of oils containing high quantities of preformed arachidonic acid and docosahexaenoic acid such as fish oils or microbial oils. If the composition is an infant formula, the total fat content preferably contributes between 30 to 55% of the total energy of the composition. In the composition, the fat source (including the LC-PUFAs such as ARA and/or DHA) preferably has a ratio of n-6 to n-3 fatty acids of about 1:2 to about 10:1, preferably about 3:1 to about 8:1.

The nutritional composition can contain vitamins and minerals understood to be essential in the daily diet and in nutritionally significant amounts. Minimum requirements have been established for certain vitamins and minerals. Non-limiting examples of minerals, vitamins and other nutrients optionally present in the nutritional composition include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L- carnitine. Minerals are usually added in salt form. The presence and amounts of specific minerals and other vitamins will vary depending on the intended population to whom the composition is administered.

The nutritional composition may contain emulsifiers and stabilizers such as soy lecithin, citric acid esters of mono- and di-glycerides, and the like. Furthermore, the nutritional composition may optionally contain other substances which may have a beneficial effect such as lactoferrin, nucleotides, nucleosides, and the like. However, in an embodiment, the nutritional composition does not contain any carotenoids.

In an embodiment, the nutritional compositions comprise a prebiotic in addition to the 2-fucosylated oligosaccharide. A prebiotic is a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Prebiotics are, for example, defined by Glenn Gibson et al., “Dietary Modulation of the Human Colonic Microbiota: Introducing the Concept of Prebiotics,” J. Nutr., 125: 1401-1412 (1995).

Non-limiting examples of prebiotics include acacia gum, alpha glucan, arabinogalactans, beta glucan, dextrans, fructooligosaccharides (FOS), galactooligosaccharides (GOS), fucosyllactoses, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof The milk oligosaccharides may be bovine's milk oligosaccharides (BMOs) and/or human milk oligosaccharides (HMOs).

In an embodiment, the nutritional composition comprises the prebiotic in an amount from 0.3 to 10% by weight of the composition. In an embodiment, the prebiotic comprises FOS. FOS may benefit immune function. To obtain the best results from FOS, however, daily intake should range between 5 and 10 grams per day as dosages above 15 grams per day may cause gas or intestinal cramping from excess Bifidobacteria populations.

A combination of prebiotics may be used such as 90% GOS with 10% short chain FOS such as the product sold under the trademark Beneo® P95, or 10% inulin such as the product sold under the trademark Beneo® HP, ST or HSI. An example of a useful prebiotic is a mixture of galacto-oligosaccharide(s), N-acetylated oligosaccharide(s) and sialylated oligosaccharide(s) in which the N-acetylated oligosaccharide(s) includes 0.5 to 4.0% of the oligosaccharide mixture, the galacto-oligosaccharide(s) includes 92.0 to 98.5% of the oligosaccharide mixture and the sialylated oligosaccharide(s) includes 1.0 to 4.0% of the oligosaccharide mixture. In an embodiment, the nutritional composition contains from 2.5 to 15.0 wt % of this prebiotic mixture on a dry matter basis, with the composition comprising at least 0.02 wt % of an N-acetylated oligosaccharide, at least 2.0 wt % of a galacto-oligosaccharide and at least 0.04 wt % of a sialylated oligosaccharide.

Suitable N-acetylated oligosaccharides include GalNAcα1,3Galβ1,4Glc and Galβ1,6GalNAcα1,3Galβ1,4Glc. The N-acetylated oligosaccharides may be prepared by the action of glucosaminidase and/or galactosaminidase on N-acetyl-glucose and/or N-acetyl galactose. Equally, N-acetyl-galactosyl transferases and/or N-acetyl-glycosyl transferases may be used for this purpose. The N-acetylated oligosaccharides may also be produced by fermentation technology using respective enzymes (recombinant or natural) and/or microbial fermentation. In the latter case, the microbes may either express their natural enzymes and substrates or may be engineered to produce respective substrates and enzymes. Single microbial cultures or mixed cultures may be used. N-acetylated oligosaccharide formation can be initiated by acceptor substrates starting from any degree of polymerisation (DP) from DP=1 onwards. Another option is the chemical conversion of keto-hexoses (e.g. fructose) either free or bound to an oligosaccharide (e.g., lactulose) into N-acetylhexosamine or an N-acetylhexosamine containing oligosaccharide as described in Wrodnigg, T. M, et al., Angew. Chem. Int. Ed. 38:827-828 (1999).

Suitable galacto-oligosaccharides include Galβ1,6Gal, Galβ1,6Galβ1,4Glc Galβ1,6Galβ1,6Glc, Galβ1,3Galβ1,3Glc, Galβ1,3Galβ1,4Glc, Galβ1,6Galβ1,6Galβ1,4Glc, Galβ1,6Galβ1,3Galβ1,4Glc Galβ1,3Galβ1,6Galβ1,4Glc, Galβ1,3Galβ1,3Galβ1,4Glc, Galβ1,4Galβ1,4Glc and Galβ1,4Galβ1,4Galβ1,4Glc. Synthesized galacto-oligosaccharides such as Galβ1,6Galβ1,4Glc Galβ1,6Galβ1,6Glc, Galβ1,3Galβ1,4Glc, Galβ1,6Galβ1,6Galβ1,4Glc, Galβ1,6Galβ1,3Galβ1,4Glc and Galβ1,3Galβ1,6Galβ1,4Glc, Galβ1,4Galβ1,4Glc and Galβ1,4Galβ1,4Galβ1,4Glc and mixtures thereof are commercially available under the trade marks Vivinal® and Elix′ or®. Other suppliers of oligosaccharides are Dextra Laboratories, Sigma-Aldrich Chemie GmbH, and Kyowa Hakko Kogyo Co., Ltd. Alternatively, specific glycoslytransferases, such as galactosyltransferases may be used to produce neutral oligosaccharides.

Suitable sialylated oligosaccharides include NeuAcα2,3Galβ1,4Glc and NeuAcα2,6Galβ1,4Glc. These sialylated oligosaccharides may be isolated by chromatographic or filtration technology from a natural source such as animal milks. Alternatively, they may also be produced by biotechnology using specific sialyltransferases either by enzyme based fermentation technology (recombinant or natural enzymes) or by microbial fermentation technology. In the latter case microbes may either express their natural enzymes and substrates or may be engineered to produce respective substrates and enzymes. Single microbial cultures or mixed cultures may be used. Sialyl-oligosaccharide formation can be initiated by acceptor substrates starting from any degree of polymerization (DP) from DP=1 onwards.

The nutritional composition may be prepared in any suitable manner For example, the nutritional composition may be prepared by blending together the protein source, the carbohydrate source, and the fat source in appropriate proportions. If used, the emulsifiers may be included at this point. The vitamins and minerals may be added at this point but are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently about 50° C. to about 80° C. to aid dispersal of the ingredients. Commercially available liquefiers may be used to form the liquid mixture. The 2-fucosylated oligosaccharide and any FOS may be added at this stage. The liquid mixture is then homogenized; for example in two stages.

The liquid mixture may then be thermally treated to reduce bacterial loads, by rapidly heating the liquid mixture to a temperature in the range of about 80° C. to about 150° C. for about 5 seconds to about 5 minutes, for example. This may be carried out by steam injection, autoclave or by heat exchanger; for example a plate heat exchanger.

Then, the liquid mixture may be cooled to about 60° C. to about 85° C.; for example by flash cooling. The liquid mixture may then be again homogenized; for example in two stages at about 10 MPa to about 30 MPa in the first stage and about 2 MPa to about 10 MPa in the second stage. The homogenized mixture may then be further cooled to add any heat sensitive components; such as vitamins and minerals. The pH and solids content of the homogenized mixture are conveniently adjusted at this point.

When the nutritional composition is a powder such as infant formula, the homogenized mixture is then transferred to a suitable drying apparatus such as a spray drier or freeze drier and converted to powder. The powder should have a moisture content of less than about 5% by weight. The 2-fucosylated oligosaccharide may be added at this stage by dry-mixing.

In another embodiment, the composition may be a supplement including 2-fucosylated oligosaccharide in an amount sufficient to achieve the desired effect in an individual. This form of administration is more suited to older children and adults. The supplement may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins or the like), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like.

The supplement can be added in a product acceptable to the consumer (who is a human or an animal), such as an ingestible carrier or support, respectively. Examples of such carriers or supports are a pharmaceutical or a food or a pet food composition. Non-limiting examples for such compositions are milk, yogurt, curd, cheese, fermented milks, milk based fermented products, fermented cereal based products, milk based powders, human milk, preterm formula, infant formula, oral supplement, and tube feeding.

Further, the supplement may contain an organic or inorganic carrier material suitable for oral or enteral administration as well as vitamins, minerals trace elements and other micronutrients in accordance with the recommendations of government bodies such as the USRDA.

The invention also relates to the following items:

1. A method for treating an influenza infection, the method comprising administering to an individual having the influenza infection a nutritional composition comprising a 2-fucosylated oligosaccharide.

2. The method of item 1 wherein the nutritional composition comprises at least 0.1 g of the 2-fucosylated oligosaccharide/100 g of the composition.

3. The method of item 1 wherein the nutritional composition comprises a prebiotic in an amount of from 0.3 to 10% by weight of the composition.

4. The method of item 3 wherein the prebiotic comprises fructooligosaccharides (FOS).

5. The method of item 1 wherein the individual is an infant or a young child.

6. The method of item 1 wherein the nutritional composition is selected from the group consisting of an infant formula, a starter infant formula, a follow-on formula, a baby food, an infant cereal composition and a fortifier.

7. The method of item 1 wherein the influenza infection is from a virus selected from the group consisting of Influenzavirus A, Influenzavirus B, Influenzavirus C, Isavirus, Thogotovirus, and combinations thereof.

8. A method for preventing an influenza infection, the method comprising administering to an individual a nutritional composition comprising a 2-fucosylated oligosaccharide.

9. The method of item 8 wherein the nutritional composition comprises fructooligosaccharides (FOS).

10. A method for improving resistance to an influenza virus in an individual, the method comprising administering to the individual a nutritional composition comprising a 2-fucosylated oligosaccharide.

11. A method for decreasing influenza in a lung of an individual, the method comprising administering to the individual a nutritional composition comprising a 2-fucosylated oligosaccharide.

12. A method for decreasing influenza-induced anorexia in an individual, the method comprising administering to the individual a nutritional composition comprising a 2-fucosylated oligosaccharide.

13. A method for decreasing influenza-induced pulmonary mucin secretion in an individual, the method comprising administering to the individual a nutritional composition comprising a 2-fucosylated oligosaccharide.

14. A method for decreasing influenza-induced IgG response in an individual, the method comprising administering to the individual a nutritional composition comprising a 2-fucosylated oligosaccharide.

15. A nutritional composition for treating or preventing an influenza infection, the nutritional composition comprising at least 0.1 g of a 2-fucosylated oligosaccharide/100 g of the composition.

### EXAMPLES

### Example 1

An example of the composition of an infant formula according to the present disclosure is given in the below Table 1. This composition is given by way of illustration only.

| Nutrient | per 100 kcal | per litre |
|---|---|---|
| Energy (kcal) | 100 | 670 |
| Protein (g) | 1.83 | 12.3 |
| Fat (g) | 5.3 | 35.7 |
| Linoleic acid (g) | 0.79 | 5.3 |
| α-Linolenic acid (mg) | 101 | 675 |
| Lactose (g) | 11.2 | 74.7 |
| Minerals (g) | 0.37 | 2.5 |
| Na (mg) | 23 | 150 |
| K (mg) | 89 | 590 |
| Cl (mg) | 64 | 430 |
| Ca (mg) | 62 | 410 |
| P (mg) | 31 | 210 |
| Mg (mg) | 7 | 50 |
| Mn (μg) | 8 | 50 |
| Se (μg) | 2 | 13 |
| Vitamin A (μg RE) | 105 | 700 |
| Vitamin D (μg) | 1.5 | 10 |
| Vitamin E (mg TE) | 0.8 | 5.4 |
| Vitamin K1 (μg) | 8 | 54 |
| Vitamin C (mg) | 10 | 67 |
| Vitamin B1 (mg) | 0.07 | 0.47 |
| Vitamin B2 (mg) | 0.15 | 1.0 |
| Niacin (mg) | 1 | 6.7 |
| Vitamin B6 (mg) | 0.075 | 0.50 |
| Folic acid (μg) | 9 | 60 |
| Pantothenic acid (mg) | 0.45 | 3 |
| Vitamin B12 (μg) | 0.3 | 2 |
| Biotin (μg) | 2.2 | 15 |
| Choline (mg) | 10 | 67 |
| Fe (mg) | 1.2 | 8 |
| l (μg) | 15 | 100 |
| Cu (mg) | 0.06 | 0.4 |
| Zn (mg) | 0.75 | 5 |
| 2FL (g) | 0.45 | 3 |

### Example 2

The following non-limiting example presents scientific data developing and supporting the concept of treatment or prevention of influenza infection using a 2-fucosylated oligosaccharide such as 2′-fucosyllactose.

Study Background

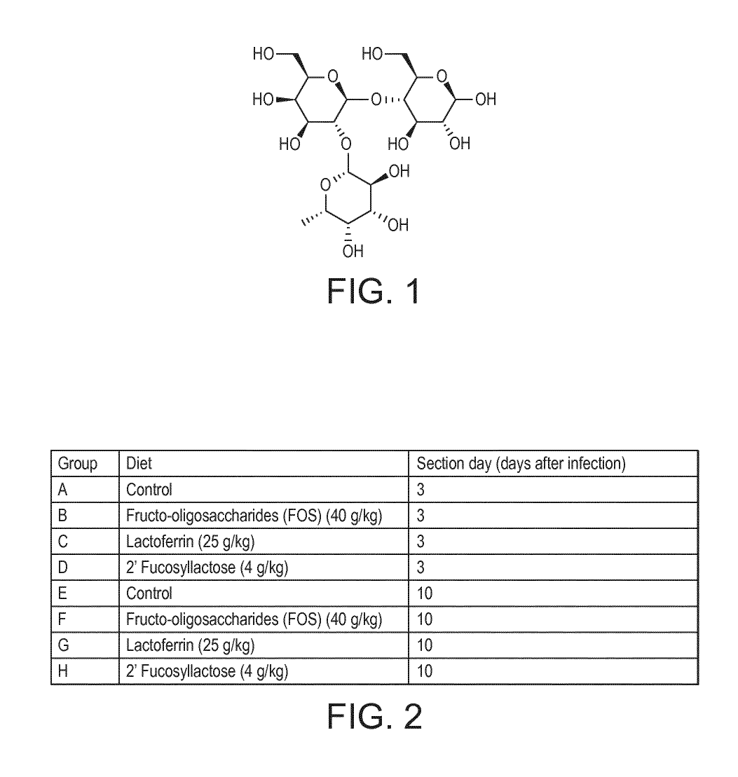

Oligosaccharides are an important constituent of human milk (the third solid component after lactose and fat). Human milk has a high content of oligosaccharides, in colostrum (20 to 23 g/L) as well as in mature milk (12 to 14 g/L). Cow's milk has an oligosaccharide content that is about 20-fold lower than that found in human milk (0.7 to 1.2 g/L in colostrum). In colostrum and mature milk, lacto-N-fucopentaose I (LNFP I) is the most abundant oligosaccharide, followed by 2′-fucosyllactose (2′-FL) (FIG. 1), lacto-N-difucotetraose, LNFP II, lacto-N-difucohexaose II, and 3-fucosyllactose (3-FL). Together these oligosaccharides account for 73% of the total weight of neutral oligosaccharides in colostrum and mature milk. Human milk oligosaccharides have an extraordinary resistance to hydrolysis by digestive enzymes of the small intestine. Human milk oligosaccharides may predominantly serve as fermentable substrates in the large intestine.

Lactoferrin is a protein of the transferrin family. Lactoferrin is present in milk, saliva, tears, bile, bloodplasma, and mucosal and genital secretions. Lactoferrin is found in large amounts in milk, where its concentration in humans may vary from 1 g/l (mature milk) to 7 g/l (colostrum). In a previous study, mice were intranasally infected with influenza virus. Mice given lactoferrin showed a significantly lower lung consolidation score on day 6 after infection compared with the control mice that were given water instead. Concurrently, the number of infiltrated leukocytes recovered from bronchoalveolar lavage fluid on day 6 was significantly lower.

Lactoferrin or 2′-fucosyllactose can potentially stimulate host defense mechanisms. Therefore, the study investigated the effects of these dietary components on the resistance of rats to an influenza infection using an infection model in which a rat-adapted Influenza A strain is used as the infectious agent.

Materials and Methods

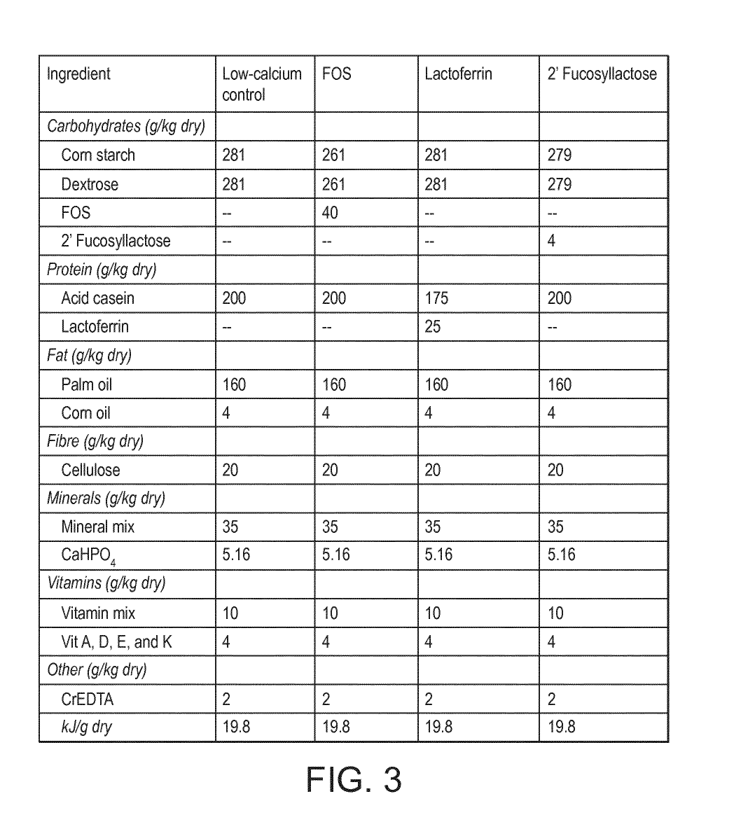

Eight-week-old male SPF Wistar rats were used. The animals were divided into eight groups of 10 animals (FIG. 2). Rats were housed at constant room temperature (20-22° C.) and relative humidity (50-60%), and a 12 h/12 h light/dark cycle. Demineralized drinking water was supplied ad libitum. Diets were supplied fresh daily. Diets contained either fructo-oligosaccharides (40 g/kg), lactoferrin (25 g/kg) or 2′-fucosyllactose (4 g/kg). All experimental diets contained 30 mmol calcium phosphate/kg. The exact composition of the diet is presented in FIG. 3. The composition of the vitamin and mineral mixtures was according to the American Institute of Nutrition 1993 (Reeves P G, Nielsen F H, Fahey G C, Jr. AIN-93 purified diets for laboratory rodents: final report of the American Institute of Nutrition ad hoc writing committee on the reformulation of the AIN-76A rodent diet. J. Nutr. 1993; 123:1939-51). In all experimental diets, total protein (200 g/kg) and fat (168 g/kg) content was similar.

Feed intake and bodyweight were measured every other day before infection and daily after infection. Delta feed intake was measured as the decrease in food intake during the entire influenza infection (10 days) corrected for the food intake at baseline (day 0).

Lungs and spleens were collected during dissection of the animals. Relative organ weights were determined as crude parameters for host responses. The number of virus particles was determined in lung tissue at 3 days after the last infection. To this end, lung tissue was homogenized in PBS and centrifuged to precipitate the debris. The supernatant was used to isolate viral genomic RNA and subsequently produce cDNA with a commercial kit. Then, cDNA was quantified by real-time PCR. Several dilutions of a known reference sample were used as a calibration line to determine the PFU. Results are expressed as plaque-forming units equivalents (PFU-eq).

Mucins are goblet cell-derived proteins containing sialic acid residues that are recognized for their ability to aggregate and inhibit hemagglutinin activity of influenza virus. In addition, mucins also assist in reducing the oxidant response of neutrophils caused by viral infection. Therefore, mucins are a component of the host-derived protective response against viral infection and subsequent pathologic sequelae.

Mucins were quantified in lung homogenates. After heat-inactivation of glycosidases, and centrifugation, the supernatant was filtered and washed. The retentate was used to measure the reduced oligosaccharides which were liberated from the mucins at a high pH. Samples were subsequently incubated with 2-cyanoacetamide, and the end-product was determined fluorimetrically. Standard solutions of N-acetylgalactosamine were used to calculate the amount of oligosaccharide side-chains liberated from mucins. Therefore, lung mucins are expressed as μmol oligosaccharide equivalents14-15.

At day 10 after Influenza infection, total serum-IgG was determined as a marker for the humoral immune response using commercially available test systems.

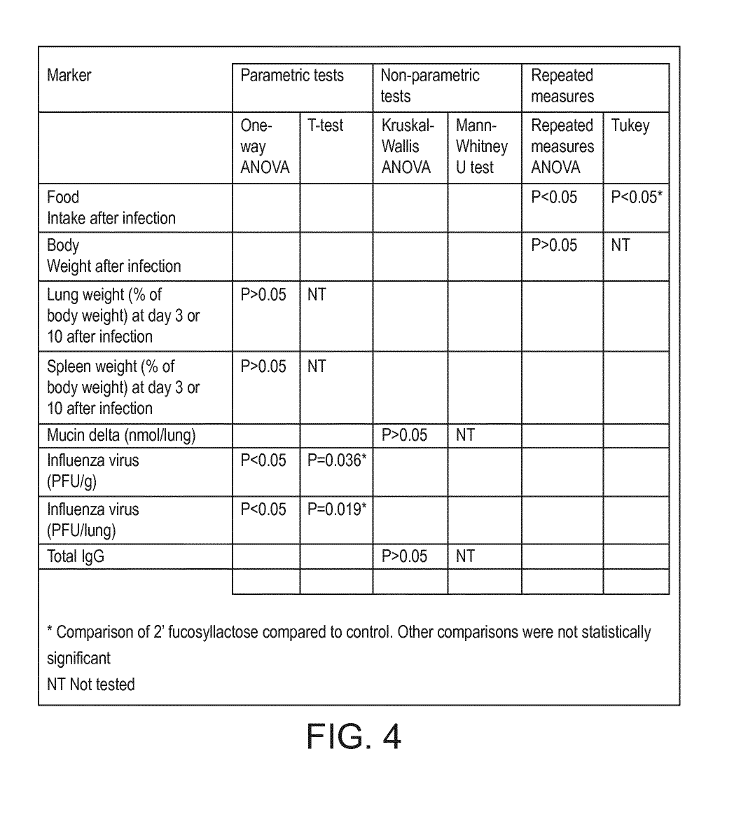

A commercially available package (Statistica 9, StatSoft Inc., Tulsa, Okla., USA) was used for all statistical analysis. All dietary groups were only compared to the control group (FIG. 4). In case of normally distributed data (as indicated by the Shapiro-Wilk test), differences between means were tested for their significance using a one-way ANOVA, followed by the Student t-test (two-sided). When data were not normally distributed, differences were tested for their significance using a Kruskall-Wallis ANOVA, followed by the non-parametric Mann-Whitney U test (two-sided). Repeated measures analysis of variance (ANOVA) was used for food intake and bodyweight data. Differences were considered statistically significant when p<0.05.

Results

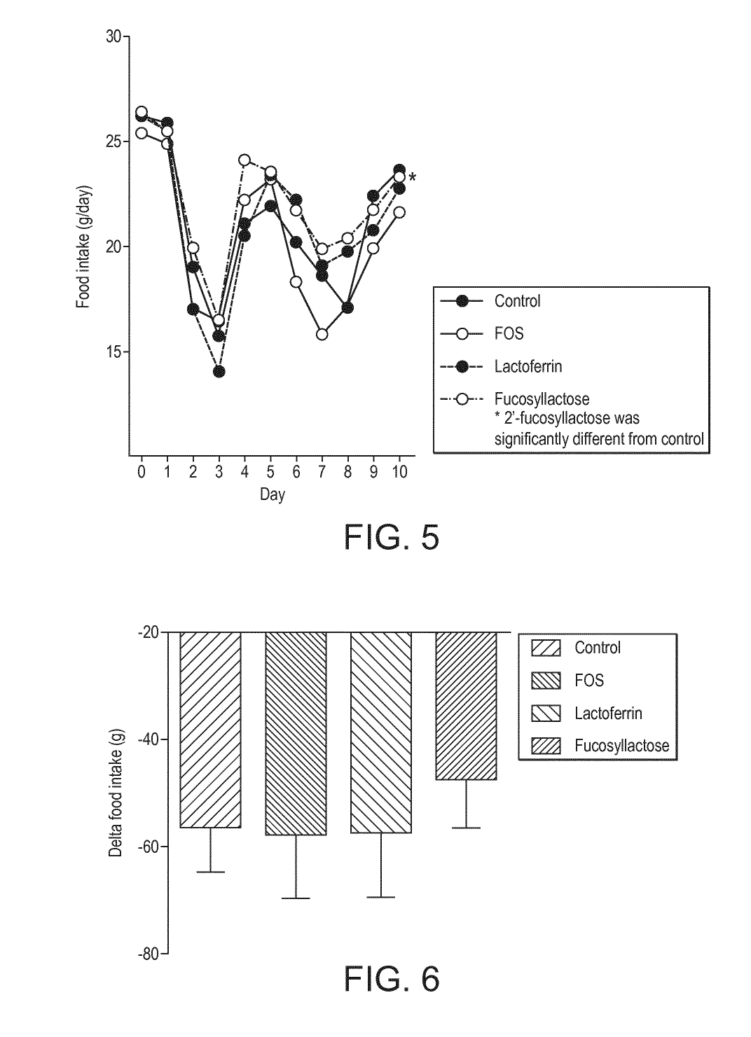

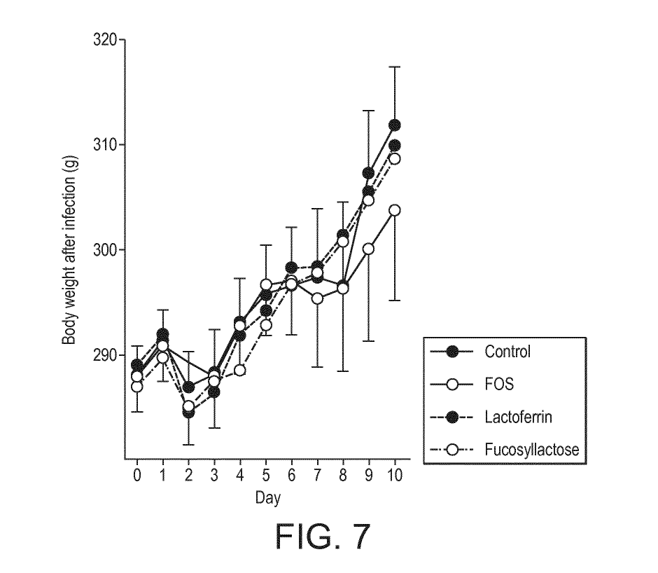

The animals were stratified based on their weight at the start of the study. As in previous influenza experiments, after infecting the animals with influenza, feed intake was reduced with approximately 10-15 grams/day (FIGS. 5 and 6). As expected, feed intake normalized after 5-6 days, but the majority of animals showed a relapse which explains the drop between 6-9 days. This relapse was observed in all previous influenza experiments. The 2′-fucosyllactose group showed decreased infection-induced anorexia (p<0.05) (FIG. 4). Growth of the animals was reduced during the first few days after infection in all animals, although it recovered thereafter (FIG. 7). There were no significant differences in infection-induced wasting between the treatment groups.

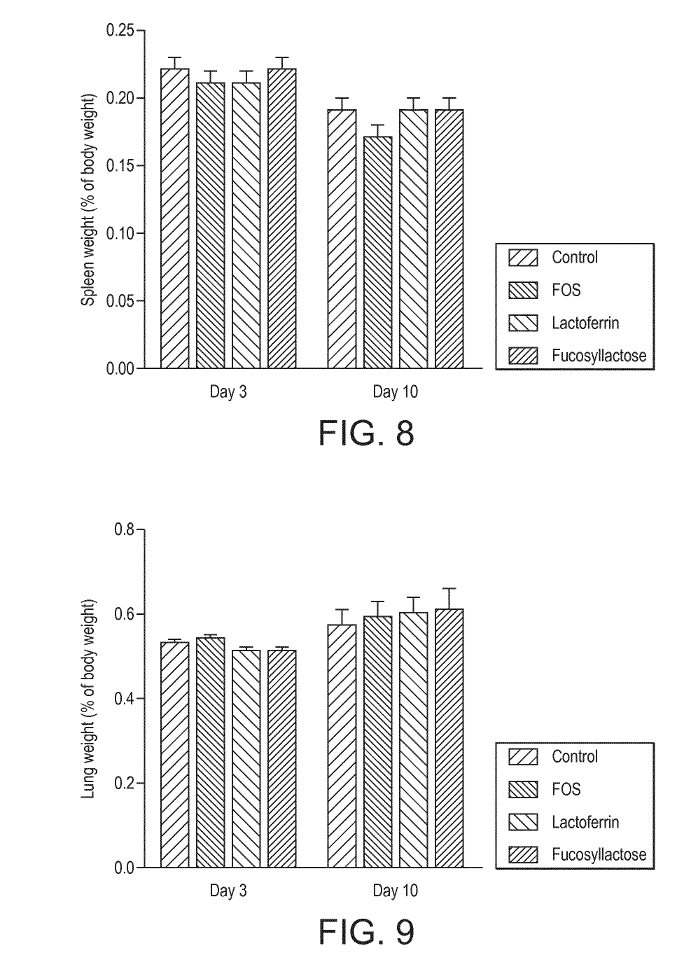

At day 3 and 10 after influenza infection both spleen and lung weights were measured as crude markers for infection. There were no significant differences in spleen weight at day 3 and 10 between the treatment groups (FIG. 8). Spleens formed about 0.18% of total body weight. Also lung weights (FIG. 9) were similar between all groups after influenza infection at day 3 and 10. Lungs formed about 0.5% of total body weight. For these parameters, no non-infected spleens and lungs were available.

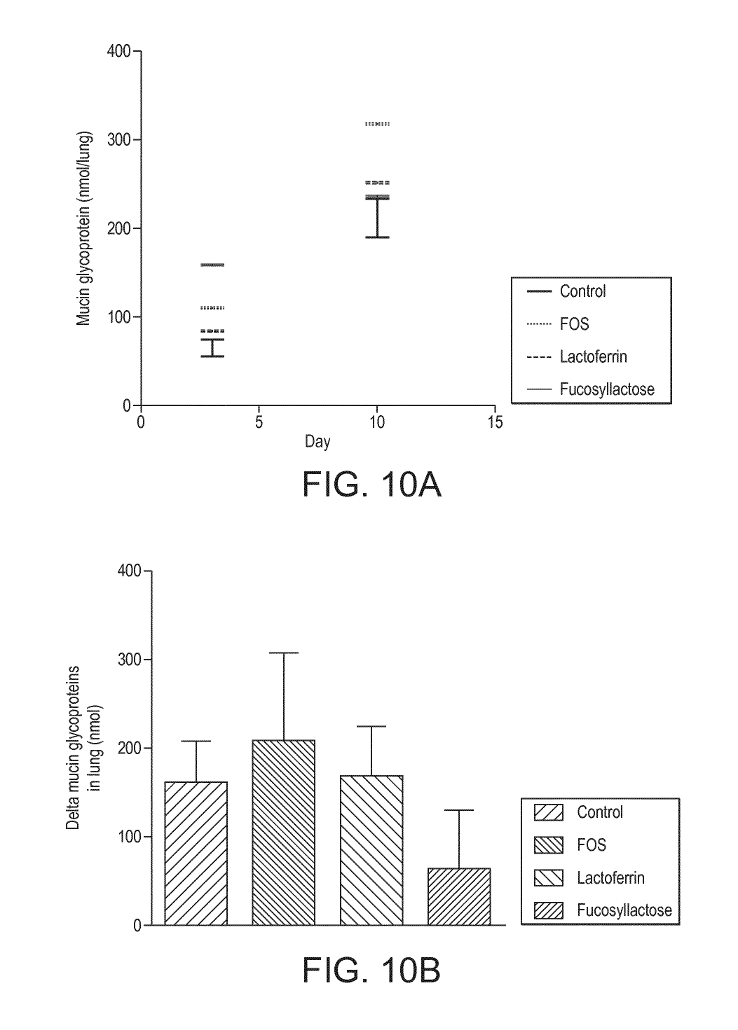

Lung mucins were measured as μmol oligosaccharide equivalents per lung. Influenza infection induced much more mucin secretion at day 10 after infection than at day 3. The mucin concentration measured at day 3 is comparable to baseline mucin concentrations in non-infected controls (data not shown). The influenza-induced increase in mucin secretion was less in the 2′-fucosyllactose group (FIGS. 10).

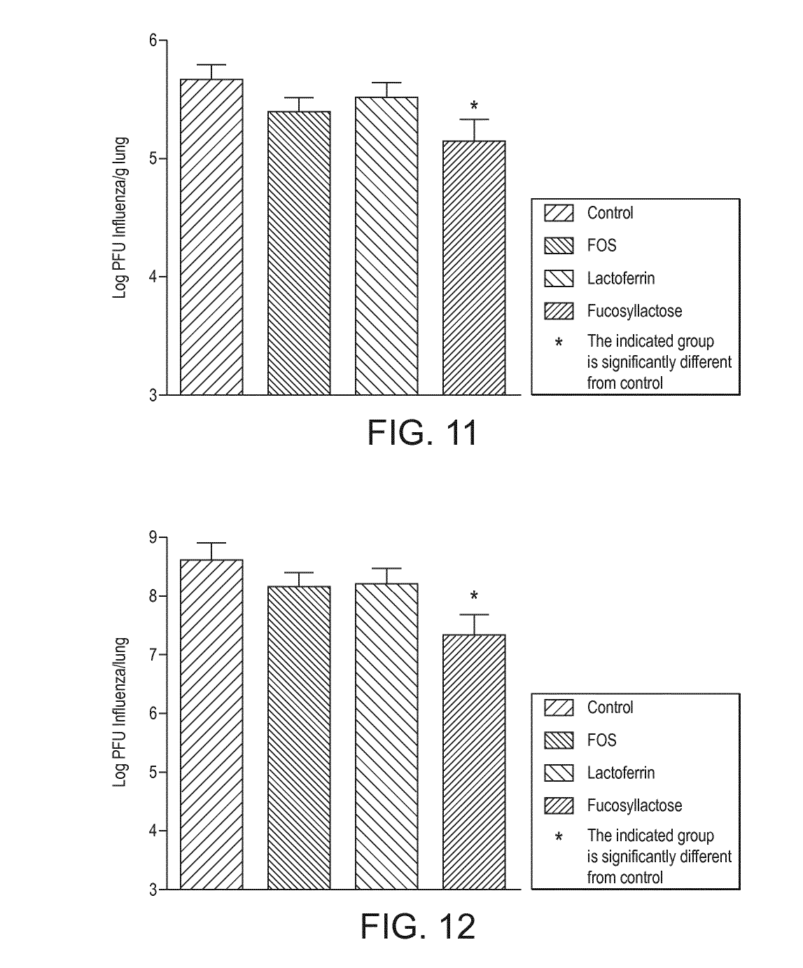

Three days after the influenza infection, rats were dissected and virus particles in the lungs were estimated by real-time PCR (FIGS. 11 and 12). 2′-fucosyllactose decreased influenza concentration (p=0.0355) and total titer (p=0.019) in the lungs, 3 days after infection (FIG. 4).

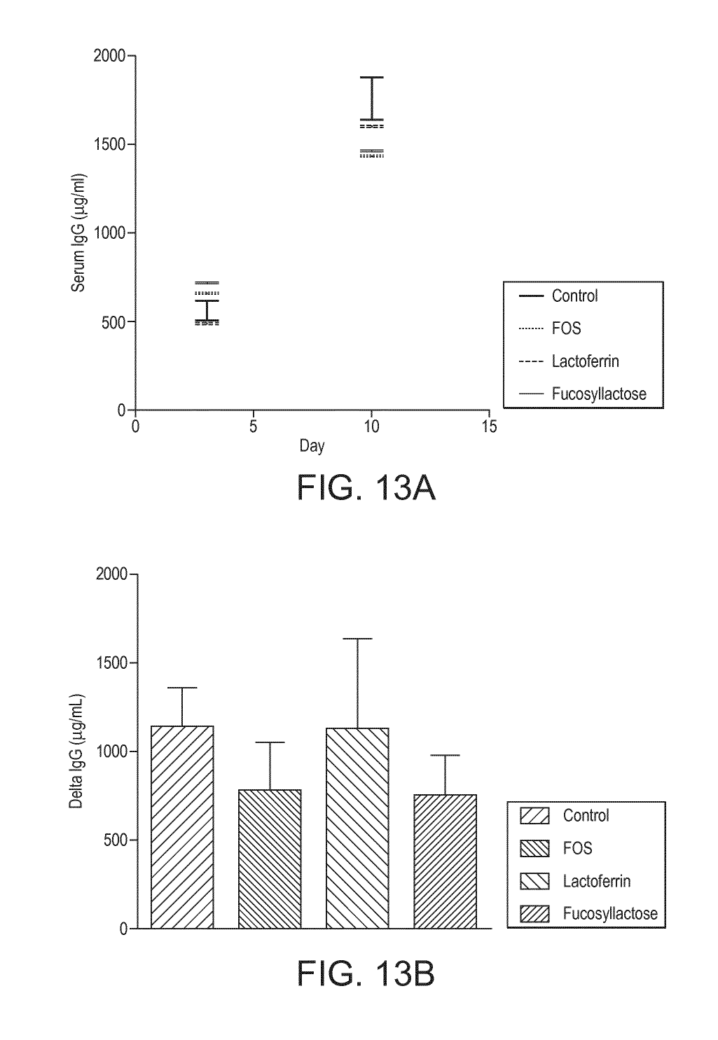

Influenza induced an increase in total serum IgG (non-specific) concentration in all dietary groups. The IgG concentration measured at day 3 is comparable to baseline IgG concentrations in non-infected controls (data not shown). The influenza-induced IgG response seemed to be less in the FOS and 2′-fucosyllactose group (FIG. 13).

Discussion

In the study, 2′-fucosyllactose improved resistance to an influenza infection in rats. This was indicated by fewer virus particles in the lung, a decreased infection-induced anorexia and a decreased pulmonary mucin secretion. Lactoferrin did not improve host defense against Influenza infection. Fructo-oligosaccharides (FOS) decreased the number of virus particles in the lung.

All human milk oligosaccharides (HMOs), like 2′ fucosyllactose, are known to be resistant to human salivary amylase, low pH in the stomach, pancreatic amylase and brush border enzymes. Less than 5% of the HMOs are digested in the intestinal tract. Hence, HMOs may play a role as prebiotics or as factors influencing the local immune system of the intestine in breast-fed infants. HMOs are one of the most important growth factors for bifidobacteria and are frequently fucosylated at their non-reducing termini Previously, a 1,2-alpha-1-fucosidase was identified from Bifidobacterium bifidum. However, in the present study, it is unknown whether the low dietary concentration of 2′-fucosyllactose used (0.4%) is able to increase bifidobacteria counts.

A previous study demonstrated that HMOs inhibit the adhesion to epithelial cells not only of common pathogens like E. coli but also other bacteria like V. cholerae and S. frris. Moreover, fucosylated mucin glycoproteins are able to bind influenza. However, a direct interaction between 2′-fucosyllactose and influenza particles in the throat seems unlikely.

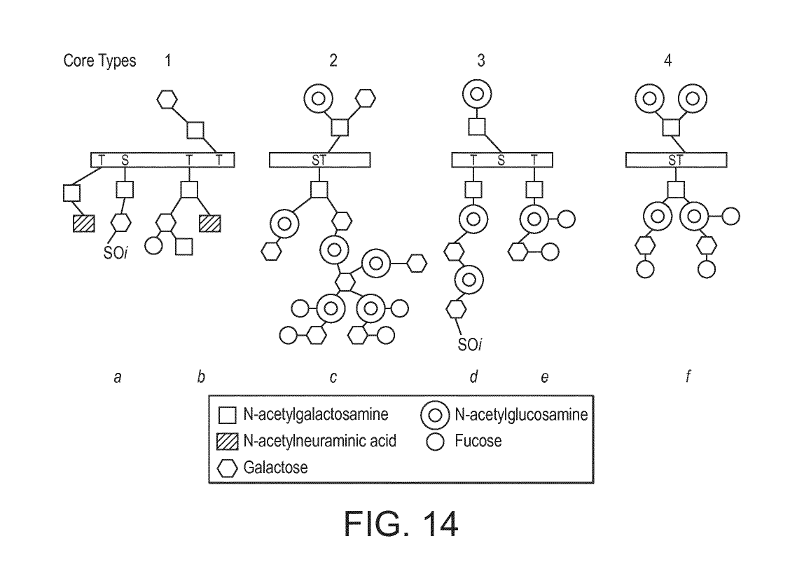

Human airway mucins represent a very broad family of polydisperse high molecular mass glycoproteins, which are part of the airway innate immunity. Human airway mucins are highly glycosylated (70-80% by weight). Mucins contribute to mucociliary defense, an innate immune defense system that protects the airways against pathogens and environmental toxins. While ONIZO-glycosylation (sugars are attached to the oxygen atom of serine or threonine) is predominant in mucins (FIG. 14), several mucins contain one or more sites for N-glycosylation (sugars are attached to the nitrogen atom of asparagine). Among the sugar residues commonly found in glycoproteins are fucose, galactose, N-acetylgalactosamine, mannose and sialic acid.

The presence of 2′-fucosyllactose in the diet could have affected mucin composition and synthesis. For instance, the modulation of the biosynthesis of the external part of N-glycans or the biosynthesis of O-glycans is controlled by diet-induced variations in the systems transferring fucose, galactose, sialic acid or hexosamines Interestingly, age-related changes in glycosylation are observed. Postnatal maturation for instance is essentially characterized by a shift from sialylation to fucosylation. Fucosylation of mucin glycoproteins could play an important role in allowing efficient virus binding to mucins.

### CONCLUSIONS

2′-fucosyllactose improves resistance to influenza in rats. It decreases influenza in the lung and influenza-induced anorexia, pulmonary mucin secretion and IgG response. Lactoferrin did not protect against influenza infection. Fructo-oligosaccharides (FOS) did decrease influenza in the lung.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for treating and/or preventing an influenza infection in an individual having the influenza infection comprising administering to the individual a nutritional composition comprising a 2-fucosylated oligosaccharide.

2. Method according to claim 1 wherein the nutritional composition comprises at least 0.1 g of the 2-fucosylated oligosaccharide/100 g of the composition.

3. Method according to claim 1 wherein the nutritional composition comprises a prebiotic in an amount of from 0.3 to 10% by weight of the composition.

4. Method according to claim 3 wherein the prebiotic comprises fructooligosaccharides (FOS).

5. Method according to claim 1 wherein the individual is an infant or a young child.

6. Method according to claim 1 wherein the nutritional composition is selected from the group consisting of an infant formula, a starter infant formula, a follow-on formula, a baby food, an infant cereal composition and a fortifier.

7. Method according to claim 1 wherein the influenza infection is from a virus selected from the group consisting of Influenzavirus A, Influenzavirus B, Influenzavirus C, Isavirus, Thogotovirus, and combinations thereof.

8. Method according to claim 1, wherein the nutritional composition comprises at least 0.1 g of a 2-fucosylated oligosaccharide/100 g of the composition.

9. (canceled)

10. Method for deceasing influenza in a lung of an individual in need of same comprising administering a nutritional composition comprising a 2-fucosylated oligosaccharide to the individual.

11. Method for decreasing influenza-induced anorexia in an individual in need of same comprising administering a nutritional composition comprising a 2-fucosylated oligosaccharide to the individual.

12. Method for decreasing influenza-induced pulmonary mucin secretion in an individual in need of same comprising administering a nutritional composition comprising a 2-fucosylated oligosaccharide to the individual.

13. Method for decreasing influenza-induced IgG response in an individual in need of same comprising administering a nutritional composition comprising a 2-fucosylated oligosaccharide to the individual.

14. Method according to claim 10 wherein the individual is an infant or a young child.

15. Method for use according to claim 10 wherein the nutritional composition is selected from the group consisting of an infant formula, a starter infant formula, a follow-on formula, a baby food, an infant cereal composition and a fortifier.

16. Method according to claim 11 wherein the individual is an infant or a young child.

17. Method according to claim 12 wherein the individual is an infant or a young child.

18. Method according to claim 13 wherein the individual is an infant or a young child.

19. Method for use according to claim 11 wherein the nutritional composition is selected from the group consisting of an infant formula, a starter infant formula, a follow-on formula, a baby food, an infant cereal composition and a fortifier.

20. Method for use according to claim 12 wherein the nutritional composition is selected from the group consisting of an infant formula, a starter infant formula, a follow-on formula, a baby food, an infant cereal composition and a fortifier.

21. Method for use according to claim 13 wherein the nutritional composition is selected from the group consisting of an infant formula, a starter infant formula, a follow-on formula, a baby food, an infant cereal composition and a fortifier.
